(19) United States
(12) Patent Application Publication
(10) Pub. No.: US 2010/0312339 A1
(43) Pub. Date: Dec. 9, 2010

(54) Method for Producing and Machining a Medical Implant as well as Implant Produced According to the Method

(76) Inventor: Hendrik John, Salzgitter-Bad (DE)

Correspondence Address: GUDRUN E. HUCKETT DRAUDT, SCHUBERTSTR. 15A, WUPPERTAL, 42289, DE

(21) Appl. No.: 12/790,867

(22) Filed: May 31, 2010

(30) Foreign Application Priority Data
May 30, 2009 (DE) 10 2009 023 239.7

(51) Int. Cl. A61F 2/02; B29C 67/20; B23Q 11/00; B26D 7/10

(52) U.S. Cl. 623/11.11; 264/46.7; 409/131; 83/15

(57) ABSTRACT

In a method for producing a medical implant with a preferably massive titanium core and a sheath of sintered open cell titanium foam, an open cell polymer foam sheath is produced with a recess adapted in size and shape to hold at least a section of a titanium core with tight fit. At least one section of the titanium core is inserted into the recess before or after the polymer foam is soaked with titanium slurry. The polymer foam sheath soaked with the titanium slurry is dried and subsequently the polymer foam is removed. The titanium foam is then sintered onto the titanium core.

### TECHNICAL FIELD OF INVENTION

In general the invention is about a process to produce and to machine a medical implant and an implant produced and machined according this process. In specific the invention is about a process to produce and to machine an implant with a preferably massive titanium core and a sheath of a sintered open cell foam, especially for manufacturing endoprosthesis, as well as an implant especially an endoprosthesis with a preferably massive titanium core and a sheath of sintered open-pore titanium foam.

### BACKGROUND OF THE INVENTION

For a few years titanium and its alloys have been used more and more for the production of implants in the orthopedic as well as dental fields, because of its properties like high strength at relatively low weight, low elasticity module, excellent biocompatibility and high corrosion resistance.

In this connection, up to now for the production of implants with a rough/porous surface, one method is e.g. that a massive core is coated via a plasma coating process, which however will result in a rough but not really three dimensional open cell surface structure. An open cell structure on the surface of the implant could get quite close to the real structure of bone, for example, which the implant is supposed to replace, and could therefore promote the ingrowth of the implant surrounding tissue into the implant.

Besides this it is known, e.g. from Textor, M.: “Titanium in medicine”, Berlin & Heidelberg: Springer-Verlag; 2001, pages 171-230, to produce porous titanium structures by replication of polymer foams and therefore to reproduce bone like structures in this way. Here the problem is however to place such a porous titanium structure permanently and with a precise fit around a massive titanium core, which is responsible for the necessary stability, to promote the ingrowth of the bone.

Another task is the freeform machining of an open cell titanium foam, like it is needed to custom fit the implant to the patient's needs. Normally such a freeform machining is done by milling or water jet cutting. During milling of titanium foam the open cell structure is normally plastically deformed due to its low stability of its filigree titanium bridges and walls, which results in a smearing and compression of the surface and the originally open cell structure is not maintained. No clean cutting edges occur and chips enter in the interior of the open cell structure, which are difficult to remove afterwards. During water jet cutting additives can enter into the structure, which are also difficult to remove.

### TASK AND SUMMARY OF THE INVENTION

The invention is based on the task to present a process to produce and machine an implant and a corresponding produced and machined implant, which avoid the disadvantages mentioned above, where the implants each consist of a preferably massive titanium core and a sintered open cell titanium foam sheath anchored fixedly on the titanium core. In particular this invention is designed to make to produce implants in the aforementioned kind, where the titanium foam sheath has a form-locking, permanent connection to the titanium core. The invention also should enable machining of the titanium foam sheath in a way that the open cell structure will remain intact at the machined surface, that clean cutting edges are produced, and the interior of the open cell structure is not contaminated by chips.

The task regarding a process to manufacture an implant is solved by a process with the attributes of the independent claims 1, 2, 3 and 4. The additional independent claim 11 concerns a process of freeform machining of the implant, the additional independent claim 16 relates to an implant produced and/or machined according to an inventive process.

Further details and advantages of the invention result from the following, solely exemplary and non-limiting description of the embodiments of the process.

### BRIEF DESCRIPTION OF THE FIGURES

FIG. 1 shows a scheme of the work flow of the implant production according of a first inventive process.

FIG. 2 shows a scheme of the work flow of the implant production according of a second inventive process.

### DESCRIPTION OF PREFERRED EMBODIMENTS

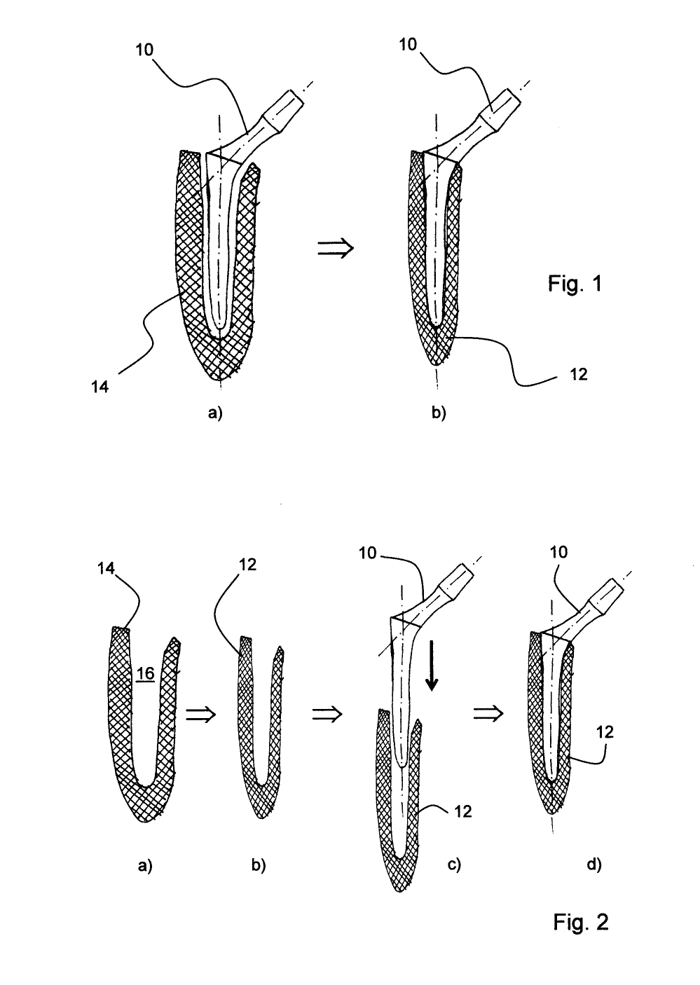

In FIG. 1 schematic process steps of a first realization of the inventive process to produce a medical implant with a preferably massive titanium core 10 and a sheath 12 of hardened open cell titanium foam in particular for the producing of an endoprosthesis are shown.

To achieve the desired permanent connection between the sheath and core, the property of shrinkage of the titanium foam during sintering is used advantageously: the titanium foam sheath is sintered onto the massive core and simultaneously shrink fitted so that the required bond with the core is created already during the production process of the titanium foam sheath.

Therefore an open cell polymeric foam sheath is produced, which works as a matrix for the titanium foam sheath and which contains a recess, in FIG. 1 not identified with a number, but in principle corresponding to the recess 16 in FIG. 2 to receive at least one section of the titanium core. Therefore the recess is adapted in size and shape to the titanium core so that the polymer foam sheath contacts closely the titanium core after introduction of the titanium core into the recess.

The titanium core 10 is then introduced into the recess, where the polymer foam sheath is saturated before or after with titanium slurry, which works as the raw material for the desired titanium foam sheath. A suitable titanium slurry would be e.g. mixture out of Ti6Al4V-powder and a binder.

Finally the excess slurry is removed by squeezing the saturated polymer foam sheath.

Because the final implant is not supposed to contain any polymer foam, it has to be removed, which could be done chemically by suitable solvents, but preferably by a pyrolysis at appr. 500° C. preferably under an argon atmosphere. Before the removal of the polymer foam the titanium slurry must be at least partially dried so that the titanium foam will not collapse during removal of the polymer foam. A suitable drying method would be e.g. that the polymer foam sheath saturated with titanium slurry is kept for 2-4 hours, preferred appr. 3 hours, at a temperature significantly higher than room temperature, in particular between 70° C. and 90° C., preferred at appr. 80° C. and afterwards will be allowed to rest for a period of 12 to 36 hours, preferred appr. 24 hours, at room temperature.

When the polymer foam is finally removed, the sintering of the titanium foam onto the core follows at conditions suitable for the used material, so e.g. over a period of appr. 2 hours at a temperature of appr. 1.250° C. under high vacuum. The result is an implant with a titanium foam sheath (FIG. 1b).

In this manufacturing method advantageously possible tolerances and non-uniformity between the inner side of the sheath and the outer side of the core are compensated by the shrinkage process.

In FIG. 2 schematic process steps are shown of the production of an implant according a second embodiment of an inventive process for production of a medical implant.

In this process variant the polymer foam sheath saturated with titanium slurry will be sintered separately. The form of the polymer foam sheath is designed with a specific oversize relative to a section of a core to be received in the titanium foam sheath that is formed later on by means of the polymer foam sheath that the shrinkage during sintering is compensated and the finally sintered titanium foam sheath can be pressed with a slight press fit onto the core.

Initially, in this variant of the process an open cell polymer foam sheath 14 with a recess 16 will be initially produced also, but which is adapted in size and shape to a section of the titanium core, so that a press fit results, when the section of the titanium core is pressed into the recess of a titanium foam sheath, which is produced when a titanium foam sheath is produced by means of the polymer foam sheath by soaking the polymer foam sheath with a titanium slurry, drying of the soaked polymer foam sheath and removing of the polymer foam sheath. The polymer foam sheath is shown in FIG. 2a.

Thereafter the polymer foam sheath will be soaked with titanium slurry and dried in the way described above, before the polymer foam for creating the titanium foam sheath 12 with a recess is removed in the way described above.

Then the titanium foam sheath is sintered under suitable conditions e.g. the conditions mentioned above. The sintered titanium foam is shown in FIG. 2b.

Finally the surface of the titanium core 10 is coated with the titanium slurry, the titanium core 10 is then pressed into the recess (FIG. 2c) of the titanium foam sheath 12 and implant created in this way is sintered. The final implant is shown in FIG. 2d.

In a modification of the process described above the titanium foam body is sintered without the recess for the titanium core. The recess will be machined e.g. by milling or drilling into the titanium foam after its completion. Thus, first the open cell polymer foam is produced without the recess. The foam will be then soaked with the titanium slurry. After drying of the polymer foam sheath soaked with the titanium slurry, the polymer foam will be removed and the thus resulting titanium foam sheath will be sintered. Finally a recess will be machined into the titanium foam sheath for at least a section of the titanium core, the recess and/or the surface of the section of the titanium core to be fitted into the recess will be coated with the titanium slurry, and the section of the titanium core will be fitted into the recess and finally sintered.

In a variation of this procedure the titanium foam sheath will be split e.g by cutting into at least two, but preferably into exactly two parts after the first sintering and before machining the recess.

In the new surfaces resulting by splitting, recesses will be machined which are partially complementary to at least one section of the titanium core, so that a recess is created to receive at least a section of the titanium core when putting the parts back together.

Then the recesses and/or the cutting areas surrounding the recesses of the sides of the parts resulting from the separation and/or the section of the titanium core are coated with titanium slurry, the section of the titanium core is placed in one of the recesses, the parts of the titanium foam sheath are put back together and the titanium foam sheath is sintered together with the titanium core. This procedure allows to machine recesses that are exactly adapted to basically any freeform shaped section of the titanium core and to then fit these sections into the recesses.

With all four above described procedures a form-locked material composite is created between the titanium core and the titanium foam sheath. In this connection, the sheath can be several millimeters thick and cover the whole core.

The implant can then be machined further for shaping, wherein advantageously one proceeds in such way that initially the open cell titanium foam is filled with a fluid, preferably water, and the fluid in the sheath is then frozen, before shaping by machining the sheath, in particular by cutting or milling, is done.

The ice of the frozen liquid fills the pores and supports therefore the filigree titanium bridges and skins/walls of the titanium foam. Therefore a precise shaping by a chipping method can be done while maintaining the open cell structure on the surface and clean cut edges on the surface.

Because the open cell pore structure is completely filled with ice, the inner pore structure is advantageously protected against contamination by chips or other particle during machining.

The freezing is preferably effect from the sheath interior to the exterior of the sheath, e.g. in that by heat is removed through the core of the implant. This has the advantage that the fluid can expand from the inside to the outside during freezing without damaging the structure of the titanium foam (“cracking” as it happens e.g. in frost damage of roads).

Advantageously one proceeds in such way that the open cell titanium foam coating is immersed a fluid-filled vessel and that freezing is carried out while the implant is immersed in the vessel. In this way, a complete filling and therewith protection of the titanium foam sheath with ice is ensured.

Depending on the kind and duration of the shaping by machining it can be advantageous to lower the environmental temperature down to the freezing point of the fluid or below in order to prevent the ice from melting and the loss of the protective action of the ice on the titanium foam sheath.

If a tool is used for shaping by machining, where a cooling fluid is used, it has to be ensured, that the freezing temperature of the cooling fluid is below the freezing temperature of the fluid which is used for filling the titanium foam sheath.

After the machining the ice is simply molten and the fluid drains from the titanium foam sheath, while advantageously causing a primary cleaning of the implant. The implant can then be cleaned and sterilized subsequently. If necessary, the titanium foam sheath can be coated at least partially with a bone cement and/or antibiotic, before the implant is implanted in the patient, wherein the outwardly open pore structure of the implant enables an optimal ingrowth of the healthy bone tissue into the implant, which predestines the implant for use without bone cement. For this purpose, the titanium foam sheath can be coated additionally with a growth-stimulating and/or anti-inflammatory material.

Within the scope of the invention numerous variations and modifications are possible, which are e.g. relating to the design of the titanium core. For example, it is possible to abrade the surface of the core before fitting it into the recess.

What is claimed is:

1-17. (canceled)

18. A method for producing a medical implant with a preferably massive titanium core and a sheath of a sintered open cell titanium foam, the method comprising the steps of:
producing an open cell polymer foam sheath with a recess adapted in size and shape to hold at least a section of a titanium core with tight fit;
soaking the polymer foam sheath with a titanium slurry,
inserting at least one section of the titanium core into the recess before or after the step of soaking with the titanium slurry,
drying the polymer foam sheath soaked with the titanium slurry,
removing the polymer foam,
sintering the titanium foam onto the titanium core.

19. The method according claim 18, comprising the step of abrading a surface of the section of the titanium core to be inserted into the recess before insertion into the recess.

20. The method according to claim 18, wherein the titanium slurry is a mixture of Ti6Al4V powder and a binder.

21. The method according to claim 18, wherein drying of the polymer foam sheath soaked with titanium slurry is carried for approximately 2 to 4 hours at a temperature of between 70 and 90° C. and subsequently for approximately 12 to 36 hours at room temperature.

22. The method according to claim 18, wherein removing of the polymer foam is done by chemical solvents or pyrolysis at around 500° C. in an argon atmosphere.

23. The method according to claim 18, wherein sintering is carried out for a period of approximately 2 hours at a temperature of approximately 1.250° C. and high vacuum.

24. A method for producing a medical implant with a preferably massive titanium core and a sheath of a sintered open cell titanium foam, the method comprising the steps of:
producing an open cell polymer foam sheath with a recess,
soaking the polymer foam sheath with a titanium slurry,
drying the polymer foam sheath soaked with the titanium slurry,
removing the polymer foam so that a titanium foam sheath with a recess is produced, wherein the recess of the polymer foam sheath is adapted in size and shape such that, after the steps of soaking, drying and removing have been performed, a section of a titanium core pressed into the recess of the titanium foam sheath creates a press fit in the recess of the titanium foam sheath,
sintering the titanium foam sheath,
coating the recess of the titanium foam sheath and/or a surface of the section of the titanium core with a titanium slurry,
pressing the section of the titanium core into the recess of the titanium foam sheath, and
sintering of the titanium foam sheath and the titanium core.

25. The method according claim 24, comprising the step of abrading a surface of the section of the titanium core to be inserted into the recess before insertion into the recess.

26. The method according to claim 24, wherein the titanium slurry is a mixture of Ti6Al4V powder and a binder.

27. The method according to claim 24, wherein drying of the polymer foam sheath soaked with titanium slurry is carried for approximately 2 to 4 hours at a temperature of between 70 and 90° C. and subsequently for approximately 12 to 36 hours at room temperature.

28. The method according to claim 24, wherein removing of the polymer foam is done by chemical solvents or pyrolysis at around 500° C. in an argon atmosphere.

29. The method according to claim 24, wherein sintering is carried out for a period of approximately 2 hours at a temperature of approximately 1.250° C. and high vacuum.

30. A method for producing a medical implant with a preferably massive titanium core and a sheath of a sintered open cell titanium foam, the method comprising the steps of:
producing an open cell polymer foam sheath,
soaking the polymer foam sheath with a titanium slurry,
drying the polymer foam sheath soaked with the titanium slurry,
removing the polymer foam,
sintering the titanium foam sheath,
creating a recess within the titanium foam sheath for receiving at least one section of the titanium core,
coating the recess and/or a surface of the section of the titanium core to be inserted into the recess with a titanium slurry,
inserting the section of the titanium core into the recess, and
sintering the titanium foam sheath and the titanium core.

31. The method according claim 30, comprising the step of abrading a surface of the section of the titanium core to be inserted into the recess before insertion into the recess.

32. The method according to claim 30, wherein the titanium slurry is a mixture of Ti6Al4V powder and a binder.

33. The method according to claim 30, wherein drying of the polymer foam sheath soaked with titanium slurry is carried for approximately 2 to 4 hours at a temperature of between 70 and 90° C. and subsequently for approximately 12 to 36 hours at room temperature.

34. The method according to claim 30, wherein removing of the polymer foam is done by chemical solvents or pyrolysis at around 500° C. in an argon atmosphere.

35. The method according to claim 30, wherein sintering is carried out for a period of approximately 2 hours at a temperature of approximately 1.250° C. and high vacuum.

36. A method for producing a medical implant with a preferably massive titanium core and a sheath of a sintered open cell titanium foam, the method comprising the steps of:
producing an open cell polymer foam sheath,
soaking the polymer foam sheath with a titanium slurry,
drying the polymer foam sheath soaked with the titanium slurry,
removing the polymer foam,
sintering the titanium foam sheath,
separating the titanium foam sheath into at least two parts,
forming partial recesses in the cut surfaces of the at least two parts, wherein the partial recesses each are partially complementary to at least one section of the titanium core and the partial recesses form a recess for receiving at least one section of the titanium core when the at least two parts are reassembled,
coating the partial recesses and/or the cut surfaces of the at least two parts, surrounding the partial recesses and/or the section of the titanium core with a titanium slurry,
inserting the section of the titanium core into one of the partial recesses,
reassembling the at least two parts of the titanium foam sheath, and
sintering the reassembled titanium foam sheath and the titanium core.

37. The method according claim 36, comprising the step of abrading a surface of the section of the titanium core to be inserted into the recess before insertion into the recess.

38. The method according to claim 36, wherein the titanium slurry is a mixture of Ti6Al4V powder and a binder.

39. The method according to claim 36, wherein drying of the polymer foam sheath soaked with titanium slurry is carried for approximately 2 to 4 hours at a temperature of between 70 and 90° C. and subsequently for approximately 12 to 36 hours at room temperature.

40. The method according to claim 36, wherein removing of the polymer foam is done by chemical solvents or pyrolysis at around 500° C. in an argon atmosphere.

41. The method according to claim 36, wherein sintering is carried out for a period of approximately 2 hours at a temperature of approximately 1.250° C. and high vacuum.

42. A method for shaping a medical implant with a massive titanium core and a sheath of sintered open cell titanium foam, comprising the steps of:
filling the open cell titanium foam sheath with a fluid,
freezing the fluid within the foam sheath,
machining the foam sheath by cutting or milling while the fluid is frozen.

43. The method according claim 42, wherein freezing is carried out from an inner core to the exterior of the sheath.

44. The method according claim 42, wherein in the freezing step heat is removed via the core of the implant.

45. The method according to claim 42, wherein the step of filling is carried out by dipping the implant into a vessel filled with the fluid and the step of freezing is carried out while the implant is within the vessel.

46. The method according to claim 42, wherein in the machining step the environmental temperature is kept at or below freezing temperature of the fluid.

47. An implant comprised of a massive titanium core and a sheath of open cell titanium foam sintered onto the massive titanium core.

48. The implant according claim 47, wherein the titanium foam is coated with growth-stimulating and/or anti-inflammatory material.

49. The implant according to claim 47, wherein the implant is shaped in that the open cell titanium foam sheath is filled with a fluid and the fluid is frozen and while the fluid is frozen the foam sheath is machined.
